(19) United States
(12) Patent Application Publication
(10) Pub. No.: US 2004/0167109 A1
(43) Pub. Date: Aug. 26, 2004

(54) Formulations of glucocorticoids to treat pathologic ocular angiogenesis

(76) Inventors: David P. Bingaman, Fort Worth, TX (US); Abbot F. Clark, Arlington, TX (US); Rajni Jani, Fort Worth, TX (US); Stella M. Robertson, Fort Worth, TX (US)

Correspondence Address: Teresa J. Schultz, Alcon Research, Ltd., 6201 South Freeway, Q-148, Fort Worth, TX, 76124-2099, US

(21) Appl. No.: 10/772,963

(22) Filed: Feb. 5, 2004

Related U.S. Application Data
(60) Provisional application No. 60/448,944, filed on Feb. 20, 2003.

(51) Int. Cl. A61K 31/58; A61K 31/573

(52) U.S. Cl. 514/176; 514/179

(57) ABSTRACT

Formulations of glucocorticoids alone and in combination with anecortave acetate are useful for preventing and treating pathologic ocular angiogenesis and associated edema.

The present invention is directed to the prevention and treatment of pathologic ocular angiogenesis. In particular, the present invention is directed to the use of certain formulations of glucocorticoids alone and in combination with anecortave acetate to treat such ocular angiogenesis.

### BACKGROUND OF THE INVENTION

There are many agents known to inhibit the formation of new blood vessels (angiogenesis or neovascularization). For example, steroids functioning to inhibit angiogenesis in the presence of heparin or specific heparin fragments are disclosed in Crum, et al., A New Class of Steroids Inhibits Angiogenesis in the Presence of Heparin or a Heparin Fragment, Science, Vol. 230:1375-1378, Dec. 20, 1985. The authors refer to such steroids as “angiostatic” steroids. Included within this class of steroids found to be angiostatic are the dihydro and tetrahydro metabolites of cortisol and cortexolone. In a follow-up study directed to testing a hypothesis as to the mechanism by which the steroids inhibit angiogenesis, it was shown that heparin/angiostatic steroid compositions cause dissolution of the basement membrane scaffolding to which anchorage dependent endothelia are attached resulting in capillary involution; see, Ingber, et al., A Possible Mechanism for Inhibition of Angiogenesis by Angiostatic Steroids: Induction of Capillary Basement Membrane Dissolution, Endocrinology, Vol. 119:1768-1775, 1986.

A group of tetrahydro steroids useful in inhibiting angiogenesis is disclosed in U.S. Pat. No. 4,975,537, Aristoff, et al. The compounds are disclosed for use in treating head trauma, spinal trauma, septic or traumatic shock, stroke, and hemorrhage shock. In addition, the patent discusses the utility of these compounds in embryo implantation and in the treatment of cancer, arthritis, and arteriosclerosis. Some of the steroids disclosed in Aristoff et al. are disclosed in U.S. Pat. No. 4,771,042 in combination with heparin or a heparin fragment for inhibiting angiogenesis in a warm blooded animal.

Compositions of hydrocortisone, “tetrahydrocortisol-S,” and U-72,745G, each in combination with a beta cyclodextrin, have been shown to inhibit corneal neovascularization: Li, et al., Angiostatic Steroids Potentiated by Sulphated Cyclodextrin Inhibit Corneal Neovascularization, Investigative Ophthalmology and Visual Science, Vol. 32(11):2898-2905, October, 1991. The steroids alone reduce neovascularization somewhat, but are not effective alone in effecting regression of neovascularization.

Tetrahydrocortisol (THF) has been disclosed as an angiostatic steroid in Folkman, et al., Angiostatic Steroids, Ann. Surg., Vol. 206(3), 1987, wherein it is suggested angiostatic steroids may have potential use for diseases dominated by abnormal neovascularization, including diabetic retinopathy, neovascular glaucoma, and retrolental fibroplasia.

Glucocorticoids have been used by the medical community to treat certain disorders of the back of the eye, in particular: Kenalog (triamcinolone acetonide), Celestone Soluspan (betamethasone sodium phosphate), Depo-Medrol (methylprednisolone acetate), Decadron (dexamethasone sodium phosphate), Decadron L. A. (dexamethasone acetate), and Aristocort (triamcinolone diacetate). These products are commonly administered via a periocular injection for the treatment of inflammatory disorders. Because of the lack of efficacious and safe therapies, there is a growing interest in using glucocorticoids for the treatment of, for example, retinal edema and age-related macular degeneration (AMD). Bausch & Lomb and Control Delivery Systems are evaluating fluocinolone acetonide delivered via an intravitreal implant for the treatment of macular edema. Oculex Pharmaceuticals is studying a dexamethasone implant for persistent macular edema. In addition, ophthalmologists are experimenting with intravitreal injection of Kenalog for the treatment of recalcitrant cystic diabetic macular edema and for exudative AMD.

Although glucocorticoids are very effective in treating many ocular conditions, there are significant side effects associated with the available products. Side effects include: endopthalmitis, cataracts, and elevated intraocular pressure (IOP). Although some side effects are due to the glucocorticoid itself, some may result from, or be exacerbated by, excipients in the formulations.

There is a need for glucocorticoid formulations that are effective in treating pathologic ocular neovascularization while causing no or lessened adverse reactions. The formulations of this invention meet that need.

### SUMMARY OF THE INVENTION

The present invention is directed to the prevention and treatment of diseases and disorders of the eye involving pathologic ocular angiogenesis using certain formulations of glucocorticoids alone and in combination with anecortave acetate.

### DETAILED DESCRIPTION OF THE INVENTION

Posterior segment neovascularization (NV) is the vision-threatening pathology responsible for the two most common causes of acquired blindness in developed countries: exudative age-related macular degeneration (AMD) and proliferative diabetic retinopathy (PDR). Currently the only approved treatments for posterior segment NV that occurs during exudative AMD is laser photocoagulation or photodynamic therapy with Visudyne®; both therapies involve occlusion of affected vasculature which results in localized laser-induced damage to the retina. For patients with PDR, surgical interventions with vitrectomy and removal of preretinal membranes are the only options currently available. No strictly pharmacologic treatment has been approved for use against posterior segment NV, although several different compounds are being evaluated clinically, including, for example, anecortave acetate (Alcon Research, Ltd.), EYE 001 (Eyetech), and rhuFabV2 (Genentech) for AMD and LY333531 (Lilly) and Fluocinolone (Bausch & Lomb) for exudative AMD and/or diabetic macular edema.

Pathologic ocular angiogenesis, which includes posterior segment NV, occurs as a cascade of events that progress from an initiating stimulus to the formation of abnormal new capillaries. The inciting cause in both exudative AMD and PDR is still unknown, however, the elaboration of various proangiogenic growth factors appears to be a common stimulus. Soluble growth factors, such as vascular endothelial growth factor (VEGF), basic fibroblast growth factor (bFGF or FGF-2), insulin-like growth factor 1 (IGF-1), etc., have been found in tissues and fluids removed from patients with pathologic ocular angiogenesis. Following initiation of the angiogenic cascade, the capillary basement membrane and extracellular matrix are degraded and capillary endothelial cell proliferation and migration occur. Endothelial sprouts anastomose to form tubes with subsequent patent lumen formation. The new capillaries commonly have increased vascular permeability or leakiness due to immature barrier function, which can lead to tissue edema. Differentiation into a mature capillary is indicated by the presence of a continuous basement membrane and normal endothelial junctions between other endothelial cells and pericytes; however, this differentiation process is often impaired during pathologic conditions.

An effective pharmacologic therapy for pathologic ocular angiogenesis and any associated edema would provide substantial efficacy to the patient, thereby avoiding invasive surgical or damaging laser procedures. Effective treatment of the pathologic ocular angiogenesis and edema would improve the patient's quality of life and productivity within society. Also, societal costs associated with providing assistance and health care to the blind could be dramatically reduced.

According to the methods of the present invention, a composition comprising a glucocorticoid alone or in combination with anecortave acetate in a pharmaceutically acceptable carrier for local administration is administered to a mammal in need thereof. The compositions are formulated in accordance with methods known in the art for the particular route of administration desired.

Glucocorticoids which may be employed in the present invention include all pharmaceutically acceptable compounds. The preferred glucocorticoids include, dexamethasone, fluoromethalone, medrysone, betamethasone, triamcinolone, triamcinolone acetonide, prednisone, prednisolone, hydrocortisone, rimexolone, and pharmaceutically acceptable salts thereof. Further examples of glucocorticoids include prednicarbate, deflazacort, halomethasone, tixocortol, prednylidene (21-diethylaminoacetate), prednival, paramethasone, methylprednisolone, meprednisone, mazipredone, isoflupredone, halopredone acetate, halcinonide, formocortal, flurandrenolide, fluprednisolone, fluprednidine acetate, fluperolone acetate, fluocortolone, fluocortin butyl, fluocinonide, fluocinolone acetonide, flunisolide, flumethasone, fludrocortisone, fluclorinide, enoxolone, difluprednate, diflucortolone, diflorasone diacetate, desoximetasone (desoxymethasone), desonide, descinolone, cortivazol, corticosterone, cortisone, cloprednol, clocortolone, clobetasone, clobetasol, chloroprednisone, cafestol, budesonide, beclomethasone, amcinonide, allopregnane acetonide, alclometasone, 21-acetoxypregnenolone, tralonide, diflorasone acetate, deacylcortivazol, RU-26988, budesonide, and deacylcortivazol oxetanone. All of the above-cited glucocorticoids are known compounds. Further information about the compounds may be found for example, in The Merck Index, Eleventh Edition (1989), and the publications cited therein, the entire contents of which are hereby incorporated in the present specification by reference.

Preferred steroids for treating pathologic ocular angiogenesis are less potent than many of the marketed products. For example, prednisolone, prednisolone acetate, rimexolone, fluoromethalone, and fluoromethalone acetate would be useful in such a scenario, but with reduced incidence of cataracts and/or elevated IOP.

The improved formulations can be delivered by intravitreal, posterior juxtascleral, or subconjunctival injection as well as via an implanted device as further below described. All cited patents are herein incorporated by reference.

Particularly preferred implanted devices include: various solid and semi-solid drug delivery implants, including both non-erodible, non-degradable implants, such as those made using ethylene vinyl acetate, and erodible or biodegradable implants, such as those made using polyanhydrides or polylactides. Drug delivery implants, particularly ophthalmic drug delivery implants are generally characterized by at least one polymeric ingredient. In many instances, drug delivery implants contain more than one polymeric ingredient.

For example, U.S. Pat. No. 5,773,019 discloses implantable controlled release devices for delivering drugs to the eye wherein the implantable device has an inner core containing an effective amount of a low solubility drug covered by a non-bioerodible polymer coating layer that is permeable to the low solubility drug.

U.S. Pat. No. 5,378,475 discloses sustained release drug delivery devices that have an inner core or reservoir comprising a drug, a first coating layer which is essentially impermeable to the passage of the drug, and a second coating layer which is permeable to the drug. The first coating layer covers at least a portion of the inner core but at least a small portion of the inner core is not coated with the first coating layer. The second coating layer essentially completely covers the first coating layer and the uncoated portion of the inner core.

U.S. Pat. No. 4,853,224 discloses biodegradable ocular implants comprising microencapsulated drugs for implantation into the anterior and/or posterior chambers of the eye. The polymeric encapsulating agent or lipid encapsulating agent is the primary element of the capsule.

U.S. Pat. No. 5,164,188 discloses the use of biodegradable implants in the suprachoroid of an eye. The implants are generally encapsulated. The capsule, for the most part, is a polymeric encapsulating agent. Material capable of being placed in a given area of the suprachoroid without migration, “such as oxycel, gelatin, silicone, etc.” can also be used.

U.S. Pat. No. 6,120,789 discloses the use of a non-polymeric composition for in situ formation of a solid matrix in an animal, and use of the composition as a medical device or as a sustained release delivery system for a biologically-active agent, among other uses. The composition is composed of a biocompatible, non-polymeric material and a pharmaceutically acceptable, organic solvent. The non-polymeric composition is biodegradable and/or bioerodible, and substantially insoluble in aqueous or body fluids. The organic solvent solubilizes the non-polymeric material, and has a solubility in water or other aqueous media ranging from miscible to dispersible. When placed into an implant site in an animal, the non-polymeric composition eventually transforms into a solid structure. The resulting implant provides a system for delivering a pharmaceutically effective active agent to the animal. According to the '789 patent, suitable organic solvents are those that are biocompatible, pharmaceutically acceptable, and will at least partially dissolve the non-polymeric material. The organic solvent has a solubility in water ranging from miscible to dispersible. The solvent is capable of diffusing, dispersing, or leaching from the composition in situ into aqueous tissue fluid of the implant site such as blood serum, lymph, cerebral spinal fluid (CSF), saliva, and the like. According to the '789 patent, the solvent preferably has a Hildebrand (HLB) solubility ratio of from about 9-13 (cal/cm3)1/2 and it is preferred that the degree of polarity of the solvent is effective to provide at least about 5% solubility in water.

Polymeric ingredients in erodible or biodegradable implants must erode or degrade in order to be transported through ocular tissues and eliminated. Low molecular weight molecules, on the order of 4000 or less, can be transported through ocular tissues and eliminated without the need for biodegradation or erosion.

Another implantable device that can be used to deliver formulations of the present invention is the biodegradable implants described in U.S. Pat. No. 5,869,079.

For posterior juxtascleral delivery of a formulation of the present invention, the preferred device is disclosed in commonly owned U.S. Pat. No. 6,413,245 B1 (cannula). Other preferred devices for delivery are disclosed in other commonly owned patents and patent applications: U.S. Pat. No. 6,416,777 B1 and U.S. Pat. No. 6,413,540 B1 (device for implantation on outer surface of the sclera).

Exemplary glucocorticoid formulations which serve the purpose of the present invention are specifically shown below in Examples 1-7. The suspensions may be delivered as previously described. The formulations of the present invention can include other non-ionic surfactants than tyloxapol, e.g., polysorbates, also known as Tweens, pluronics, and Spans. Ionic surfactants can also be used, e.g., sodium lauryl sulfate or anionic bile salts. Amphoteric surfactants, such as, lecithin and hydrogenated lecithin can be used. The pH can vary from 5.0-8.4, but is preferably about 6.8-7.8. Other appropriate buffer systems, such as, citrate or borate can be employed in the present formulations. Different osmolality adjusting agents can also be used, such as, potassium chloride, calcium chloride, glycerin, dextrose, or mannitol.

### EXAMPLE 1

Triamcinolone Acetonide Sterile Suspension

| Ingredient | Concentration w/v % |
|---|---|
| Triamcinolone Acetonide | 0.4-2.0% |
| Monobasic Sodium Phosphate Diltydrate | 0.051% |
| Dibasic Sodium Phosphate Dodecahydrate | 0.5% |
| Tyloxapol | 0.01-0.4% |
| Sodium Chloride | 0.76% |
| NaOH/HCl | pH adjust to 5.0-8.4 |
| Water for injection | q.s. 100% |

### EXAMPLE 2

Rimexolone Sterile Suspension

| Ingredient | Concentration w/v % |
|---|---|
| Rimexolone | 0.1-4.0% |
| Monobasic Sodium Phosphate Diltydrate | 0.051% |
| Dibasic Sodium Phosphate Dodecahydrate | 0.5% |
| Tyloxapol | 0.01-0.4% |
| Sodium Chloride | 0.76% |
| NaOH/HCl | pH adjust to 5.0-8.4 |
| Water for injection | q.s. 100% |

### EXAMPLE 3

Prednisolone Sterile Suspension

| Ingredient | Concentration w/v % |
|---|---|
| Prednisolone Acetate | 0.1-2.0% |
| Monobasic Sodium Phosphate Diltydrate | 0.051% |
| Dibasic Sodium Phosphate Dodecahydrate | 0.5% |
| Tyloxapol | 0.01-0.4% |
| Sodium Chloride | 0.76% |
| NaOH/HCl | pH adjust to 5.0-8.4 |
| Water for injection | q.s. 100% |

### EXAMPLE 4

Fluoromethalone Acetate Sterile Suspension

| Ingredient | Concentration w/v % |
|---|---|
| Fluoromethalone Acetate | 0.1-1.0% |
| Monobasic Sodium Phosphate Diltydrate | 0.051% |
| Dibasic Sodium Phosphate Dodecahydrate | 0.5% |
| Tyloxapol | 0.01-0.4% |
| Sodium Chloride | 0.76% |
| NaOH/HCl | pH adjust to 5.0-8.4 |
| Water for injection | q.s. 100% |

The present invention also contemplates the use of a glucocorticoid in combination with the angiostatic agent, anecortave acetate. As used herein, anecortave acetate refers to 4,9(11)-pregnadien-17α,21-diol-3,20dione-21-acetate and its corresponding alcohol is (4,9(11)-pregnadiene-17α,21-diol-3,20-dione). Presently, anecortave acetate is undergoing clinical trials for its use in persons suffering from subfoveal choroidal neovascularization secondary to AMD. A glucocorticoid alone or in combination with anecortave acetate is useful for treating persons suffering from pathologic ocular angiogenesis, in particular, exudative AMD and/or PDR, as well as subretinal or retinal edema associated with either condition. In addition to being effective in inhibiting the neovascularization associated with wet AMD and PDR, anecortave acetate is useful in controlling any IOP rise associated with the use of a glucocorticoid.

Examples of formulations of the above-described combination are shown below:

### EXAMPLE 5

| Ingredient | Concentration w/v % |
|---|---|
| Anecortave Acetate | 3% |
| Triamcinolone Acetonide | 0.5-4.0% |
| Monobasic Sodium Phosphate Diltydrate | 0.051% |
| Dibasic Sodium Phosphate Dodecahydrate | 0.5% |
| Tyloxapol | 0.05-0.4% |
| Sodium Chloride | 0.76% |
| NaOH/HCl | pH adjust to 5.0-8.4 |
| Water for injection | q.s. 100% |

### EXAMPLE 6

A typical example of topical formulation of Anecortave Acetate is as follows:

| Ingredient | Concentration w/v % (Preferred Range) |
|---|---|
| Anecortave Acetate | 0.1-6% (1-3%) |
| Polyquad | 0.0005-0.01% (0.0001%) |
| HPMC | 0.02-1.0% (0.5%) |
| Mannitol (b) | 0.0-5.0% (3.82%) |
| Sodium Chloride (d) | 0.0-0.8% (0.17%) |
| Disodium Edetate | 0.0-0.2% (0.01%) |
| Polysorbate-80 (c) | 0.005-0.4% (0.05%) |
| NaOH and/or HCl | q.s. pH 5.0-8.4 (6.8-7.8) |
| Purified Water | q.s. 100% |

(a) other suitable polymers include cellulosic polymers like HPMC, HEC, sodium CMC), polyvinyl alcohol (PVA), Polyvinyl Pyrrolidone (PVP), polyacrylamide, and other water miscible/soluble polymers to impart viscosity to the product and to stabilize suspension.

(b) both ionic as well nonionic agents are used to adjust Osmolality of the product either alone or in combination. This also stabilize the suspension.

(c) other surfactants that can be used are non-ionic (Tyloxapol, Tweens, Spans) anionic (lecithin, hydrogenated lecithins), or anionic (sodium lauryl sulfate, bile salts).

### EXAMPLE 7

### Unit Dose Composition (Preservative Free Product Packaged in Unit Dose)

| Ingredients | Concentration (Preferred Range) |
|---|---|
| Anecortave Acetate | 0.1-6% (1-3%) |
| Carbomer 974P | 0.02-0.8% (0.3%) |
| Mannitol | 0.0-5.0% (3.82%) |
| Sodium Chloride | 0.0-0.8% (0.17%) |
| Polysorbate-80 | 0.005-0.4% (0.05%) |
| NaOH/HCl | q.s. pH 4.0-8.0 (6.8-7.8) |
| Purified Water | q.s. 100% |

### EXAMPLE 8

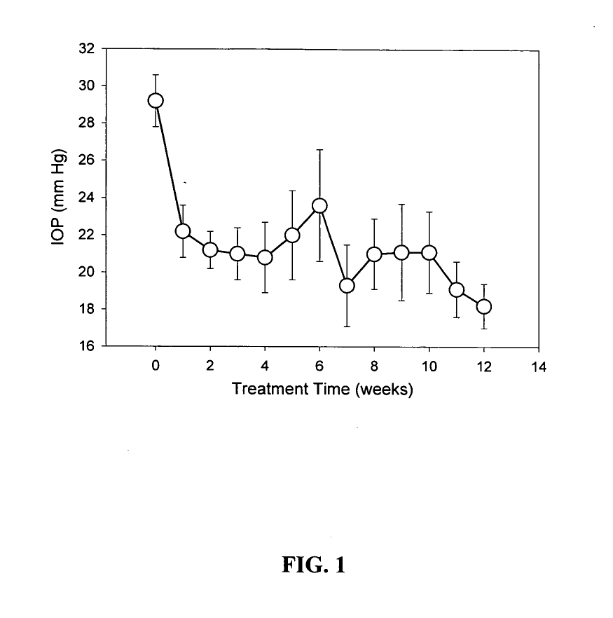

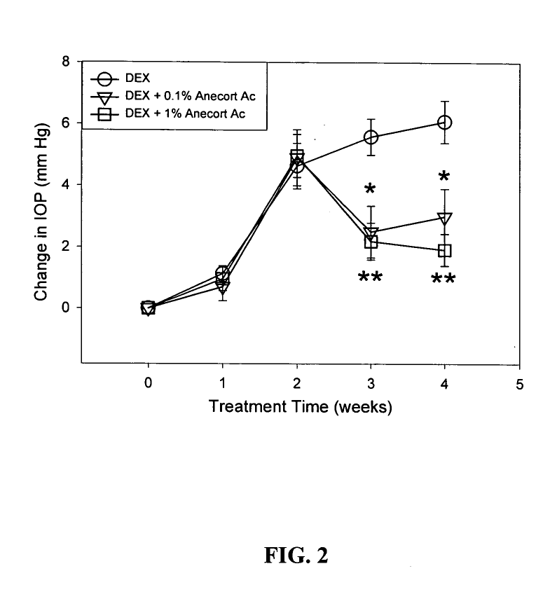

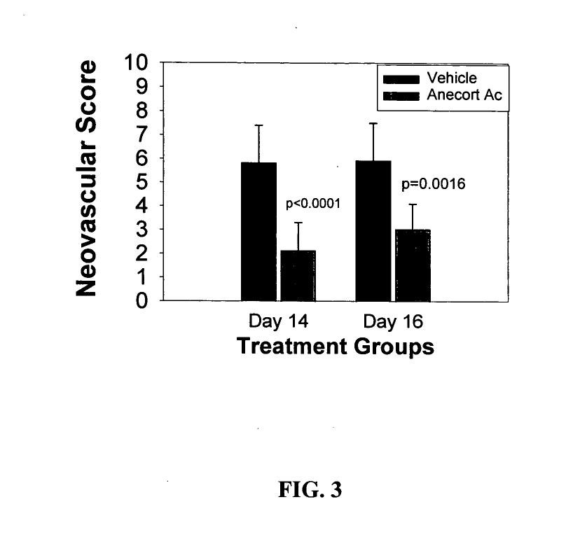

Patients (η=15) with documented glucocorticoid induced ocular hypertension were treated topically with 1% anecortave acetate eye drops three times per day for up to 12 weeks. The patients continued to receive their glucocorticoid medication. IOP was significantly reduced after anecortave acetate treatment (from 29 mm Hg to ˜19-22 mm Hg). See FIG. 1.

The compositions administered according to the present invention comprise a pharmaceutically effective amount of a glucocorticoid alone or in combination with anecortave acetate. As used herein anecortave acetate refers to 4,9(11)-pregnadien-17α,21-diol-3,20dione-21-acetate and its corresponding alcohol 4,9(11)-pregnadien-17α,21-diol-3,20dione. As used herein, a “pharmaceutically effective amount” is one which is sufficient to reduce or prevent pathologic ocular angiogenesis and any associated edema.

The preferred compositions of the present invention are intended for administration to a human patient suffering from pathologic ocular angiogenesis and/or any associated edema. Examples of diseases or disorders encompassed by pathologic ocular angiogenesis and any associated edema include, but are not limited to: age-related macular degeneration, diabetic retinopathy, chronic glaucoma, retinal detachment, sickle cell retinopathy, rubeosis iritis, uveitis, neoplasms, Fuch's heterochromic iridocyclitis, neovascular glaucoma, corneal neovascularization, neovascularization resulting from combined vitrectomy and lensectomy, retinal ischemia, choroidal vascular insufficiency, choroidal thrombosis, carotid artery ischemia, retinal artery/vein occlusion, e.g., central retinal artery occlusion and branch retinal vein occlusion, contusive ocular injury, and retinopathy of prematurity.

This invention has been described by reference to certain preferred embodiments; however, it should be understood that it may be embodied in other specific forms or variations thereof without departing from its special or essential characteristics. The embodiments described above are therefore considered to be illustrative in all respects and not restrictive, the scope of the invention being indicated by the appended claims rather than by the foregoing description.

We claim:

1. A method for treating pathologic ocular angiogenesis and any associated edema which comprises, administering a composition comprising an effective amount of a glucocorticoid.

2. The method of claim 1 wherein the composition further comprises an effective amount of anecortave acetate.
